(19) United States
(12) Patent Application Publication
(10) Pub. No.: US 2002/0035139 A1
(43) Pub. Date: Mar. 21, 2002

(54) COX-2 inhibitors and the prevention of the side effects of radiation therapy

(76) Inventors: Arthur L. Herbst, Chicago, IL (US); Ralph Weichselbaum, Chicago, IL (US)

Correspondence Address: MCDERMOTT WILL & EMERY, 600 13TH STREET, N.W., WASHINGTON, DC, 20005-3096, US

(21) Appl. No.: 09/884,466

(22) Filed: Jun. 20, 2001

Related U.S. Application Data
(60) Provisional application No. 60/212,685, filed on Jun. 20, 2000.

(51) Int. Cl. A61K 31/415

(52) U.S. Cl. 514/406

(57) ABSTRACT

A generalized method is disclosed for reducing the deleterious side effects of radiotherapy in subjects undergoing radiotherapy for the treatment of cancer. The method is the administration to a subject of a side-effect reducing amount of one or more selective cyclooxygenase-2 (COX-2) inhibitor.

This application claims priority to U.S. provisional patent application Serial No. 60/212,685, filed Jun. 20, 2000.

### FIELD OF THE INVENTION

This invention is directed toward methods of reducing the side effects associated with radiotherapy in cancer patients.

### BACKGROUND OF THE INVENTION

The acute effects of radiotherapy are a result of the interruption of rapidly dividing cell renewal systems. In the pelvis, these effects may be manifested as acute radiation proctitis, cystitis, prostatitis, as well as dermatitis. Presently, the only FDA approved intervention for acute radiation effects is the use of Ethiol, a sulfhydryl-containing compound (WR-2721), is reported to decrease the dry mouth following radiation treatment for head and neck cancer. However, Ethiol does not limit acute mucosal effects and there is concern that Ethiol may actually protect tumors. In addition, patients who receive even limited field radiotherapy may develop fatigue, which may limit their ability to tolerate treatment or to carry on daily activity.

Hallahan D E et al., Proc. Natl. Acad. Sci. USA 91(11): 4897-901 (1994) identified a signaling pathway involving the activation of phospholipase A2 and protein kinase C in human cells that confers x-ray induction of the tumor necrosis factor alpha (TNFα) gene. Phospholipase A2 is involved in the production of arachidonate, part of the prostaglandin synthesis pathway. Recently, two isoforms of the enzymes that synthesize prostaglandins have been identified. The first, COX-1, is a constitutive enzyme that has a housekeeping physiological function. The second, COX-2, is induced by diverse inflammatory stimuli, oncoproteins and growth factors. COX-2 is known to promote carcinogenesis as well as growth of established tumors and is up-regulated in the high percentage of common human cancers. Thus, COX-2 is a target for prevention as well as therapeutic intervention.

### SUMMARY OF THE INVENTION

The invention provides a method of reducing the side effects associated with radiotherapy, comprising administering to a cancer patient undergoing radiotherapy a cyclooxygenase-2 (COX-2) inhibitor. COX-2 inhibitors inhibit the acute mucosal effects of radiation, as well as associated fatigue, by blocking induction of the COX-2 proteins.

The reduced side effects can include an acute mucosal effect of radiation on the urinary or gastrointestinal tract; fatigue; diarrhea, rectal bleeding, proctitis, or sigmoiditis; urinary frequency, prostatitis, or cystitis; or dermatitis.

In one embodiment, the administered COX-2 inhibitor is rofecoxib. In another embodiment, the administered COX-2 inhibitor is celecoxib.

In one embodiment, the radiation treatment that causes the side effects is directed outside of the pelvis. In another embodiment, the radiation treatment that causes the side effects is directed to the pelvic area.

### DETAILED DESCRIPTION OF THE INVENTION

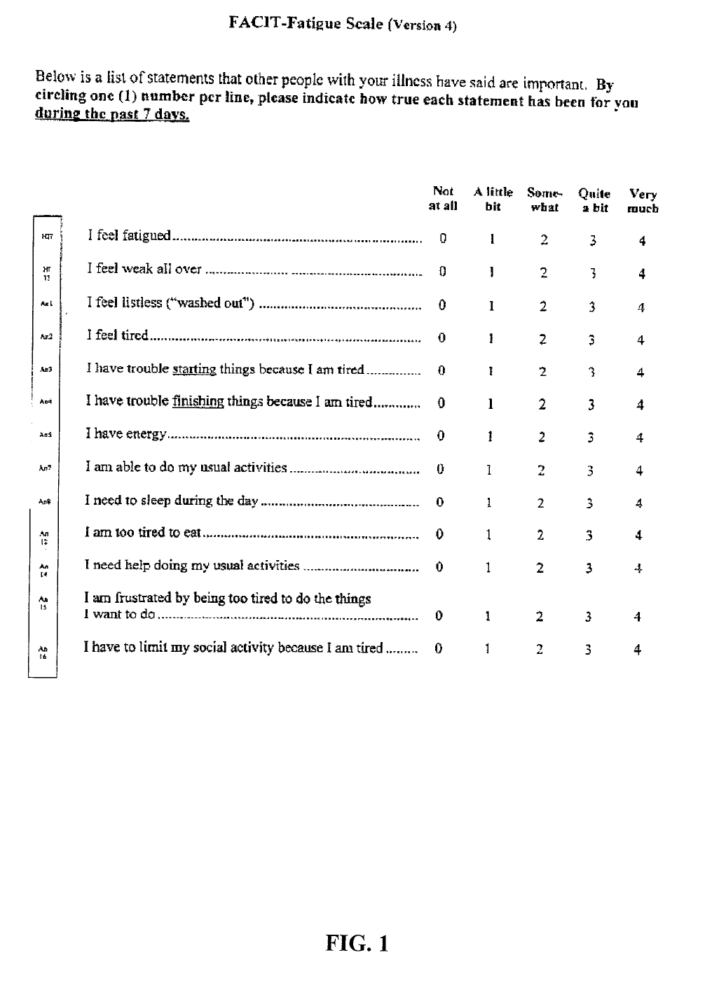

FIG. 1 is an interview form for determining fatigue (FACT-An/fatigue Scoring Guidelines).

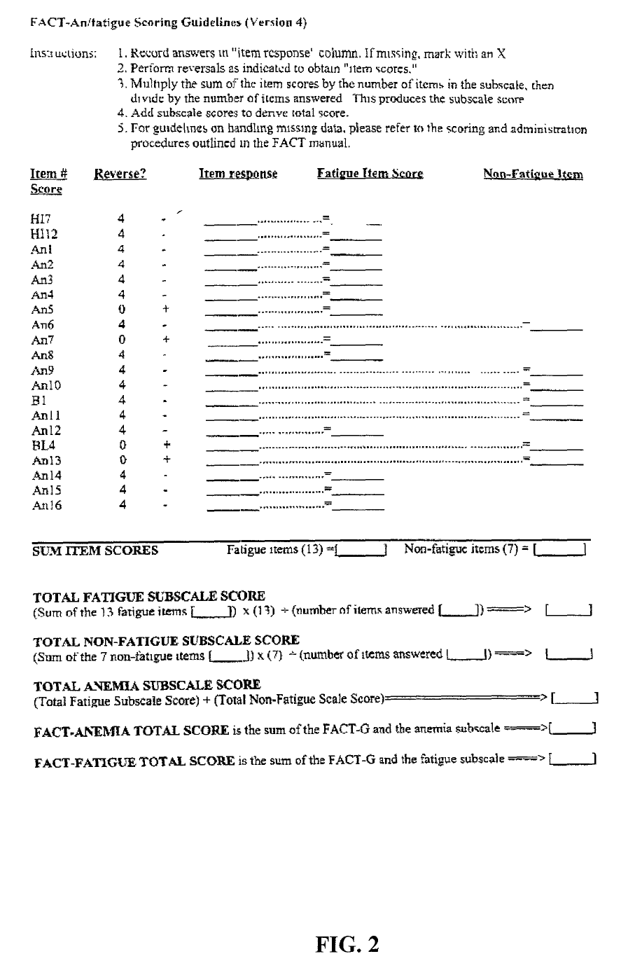

FIG. 2 is an interview form for determining fatigue (FACIT-Fatigue Scale).

### DETAILED DESCRIPTION OF THE INVENTION

The present invention originated with observations made during pelvic radiation for prostate carcinoma. Our clinical observations suggest that the use of COX-2 inhibitors, Celebrex® (Celacoxib, Searle) and Vioxx® (Rofecoxib, Merck) decrease the acute mucosal effects of radiation especially on the urinary and gastrointestinal tracts, as well as associated fatigue during radiotherapy.

COX-2 inhibitor agents given to patients receiving radiation therapy to the pelvis can reduce the frequency of diarrhea and rectal bleeding and the symptoms associated with proctitis and sigmoiditis. Administration of these COX-2 inhibitor agents can lessen urinary frequency and symptoms usually associated with prostatitis in male and cystitis in individuals of both sexes. Finally, administration of these COX-2 inhibitor agents during radiation treatment can reduce the fatigue usually experienced by patients receiving radiation and that this latter benefit will be for patients receiving treatment to any site, including those outside the pelvis.

COX-2 inhibitors decrease the acute side effects of radiotherapy. These side effects are gastrointestinal and hematological toxicity. These side effects include cystitis, proctitis, prostatitis, pneumonitis and large and small bowel irritation as reflected by diarrhea and other gastrointestinal symptoms including nausea and vomiting. Additionally, mucosal effects of radiotherapy including dermatitis, esophagitis, oral and upper airway mucositis and the dermatitis seen in the intergenous areas of the body including the breast/axillary fold, vulva/vaginal folds and large skin fold areas in the abdomen can be protected from the side effects of radiotherapy. The fatigue associated with local and large field radiotherapy can be decreased by COX-2 inhibition.

In addition, COX-2 inhibitors have been shown to potentiate the anti-tumor effects of radiotherapy, and unlike Ethiol, do not carry the concern of radioprotection. COX-2 inhibitors can be given to enhance patient well being and energy during and immediately after radiotherapy treatment and may also be given to limit acute effects of radiotherapy. Cox-2 inhibitors may replace corticosteroids as treatment for some of the intermediate forms of radiation pnuemonitis.

Background. Hallahan D E et al., “Membrane-derived second messenger regulates x-ray-mediated tumor necrosis factor alpha gene induction.” Proc. Natl. Acad. Sci. USA 91(11): 4897-901 (1994) identified a signaling pathway involving the activation of phospholipase A2 and protein kinase C in human cells that confers x-ray induction of the tumor necrosis factor alpha (TNFα) gene. Treatment of human cells with ionizing radiation or H₂O₂ was associated with the production of arachidonic acid. Inhibition of phospholipase A2 abolished radiation-mediated arachidonate production as well as, the subsequent activation of protein kinase C and tumor necrosis factor alpha gene expression. Hallahan showed that ionizing radiation-mediated gene expression in human cells is regulated in part by extranuclear signal transduction.

The arachadonic acid that is is produced is metabolized by COX-2 to various metabolic products, which are chemoattractants for inflammatory cells.

Subsequently, Steinauer K K et al., “Radiation induces up regulation of Cox-2 protein in prostate cancer cells.” Int. J. Rad. Onc. Biol. Phys. 48(2): 325-8 (2000) investigated the impact of gamma-irradiation on cyclooxygenase-2 (COX-2) expression and its enzymatic activity in PC-3 cells. Cell cycle redistribution, viability, and apoptosis were quantitated in control and irradiated cells with or without the COX-2 inhibitor NS-398. Steinauer observed a dose-dependent increase in COX-2 of following increased irradiation dosages. The prostaglandin (PGE(2)) level in irradiated cells was higher than in controls while cells irradiated in the presence of NS-398 had reduced PGE(2) levels. Steinauer found no differences in cell cycle distribution or apoptosis between cells irradiated in the presence or absence of NS-398. Thus, COX-2 protein is up-regulated and enzymatically active after irradiation, resulting in elevated levels of PGE(2). This effect can be suppressed by NS-398, which has clinical implications for therapies combining COX-2 inhibitors with radiation therapy.

The induction of proinflammatory cytokines TNFα and IL-1 following exposure of cells and animals to ionizing radiation have been reported. O'Brien-Ladner A et al., Radiat. Res. 136(1): 37-41 (1993). It is also known that cyclooxygenase inhibitors block production of these cytokines. See also, Van Der Meeren A et al. Radiat. Res. 155(6) 858-65 (2001).

Proposed Mechanism of Action; Tests. We propose that an inflammatory response mediates in part the acute mucosal intestinal, skin, lung, prostatic and bladder effects of ionizing radiation. Additionally we propose that a component of radiation induced fatigue is mediated by the inflammatory response and as reflected by acute phase reactant proteins that increase during radiotherapy.

We tested whether COX-2 inhibitors suppress TNFα production following localized radiotherapy in mice. TNFα production was not suppressed during times studied (24 hours following radiation).

Thus, COX-2 inhibitors mediate their anti-radiation induced inflammatory effects by directly inhibiting COX-2 and not indirectly by inhibiting one class of cytokines. Accordingly, the anti-inflammatory effects are not restricted to the effects of one cytokine. Some of the long term effects of radiotherapy, fibrosis, adhesions, and small volume necrosis can be inhibited or attenuated by decreasing the inflammatory effects of radiation therapy.

Utility. The side effects of radiation for prostate cancer include acute urinary side effects (see, Chou et al., Int. J. Radiat. Oncol. Biol. Phys. 47:115 (2001); Dearnaly et al, Lancet 358: 267 (1999); O'Sullivan et al., Clin. Oncol. 12: 217 (2000)). The acute urinary side effects can be at the GRI level (with frequency of nocturia approx. 2× pretreatment, with a dysuria urgency requiring no medication) or at the GRII level (with frequency of nocturia less than hourly, with a dysuria urgency requiring medication). In general, 30-40% of patients suffer at the GRI level and 20-30% of patients suffer at the GRII level. Overall 50-60% of patients have these effects, which last approximately 6 months in duration. Some patients suffer the more severe GR III or IV.

Many cancer patients are at risk for suffering the side effects of radiation treatment. TABLE 1 below shows the cancer incidence worldwide (Globocan, 2000), each of which can be treated by radiation treatment.

TABLE 1
CANCER INCIDENCE WORLDWIDE

| Cancer Type | North America Cases | N. Europe Cases | W. Europe Cases |
|---|---|---|---|
| Breast | 202,044 | 54,551 | 115,308 |
| Cervix | 14,845 | 6,049 | 13,282 |
| Uterus | 35,960 | 9,440 | 19,214 |
| Prostate | 211,950 | 37,046 | 84,856 |
| Lung | | | |
| men | 119,664 | 19,336 | 75,350 |
| women | 85,944 | 18,063 | 18,183 |
| Colorectal | | | |
| men | 83,777 | 26,409 | 61,128 |
| women | 80,896 | 24,953 | 58,255 |
| Head & Neck | | | |
| men | 13,731 | 3,746 | 17,026 |
| women | 8,914 | 1,980 | 5,119 |
| TOTAL | 857,725 | 201,573 | 467,721 |
| TOTAL IN NORTH | | | 1,527,019 |
| AMERICA AND | | | |
| EUROPE | | | |

The method of the invention provides substantial benefit to the patient population as a reduction in side-effects of 25% to 75% is achieved. The sample size estimation for each group (treatment and control) is provided in TABLE 2.

TABLE 2
SAMPLE SIZE EXAMPLES

| | Prevalence of side | Prevalence of size |
|---|---|---|
| | effects = | effects = |
| | 60% | 75% |
| Expected reduction = 25% | 60 --> 45% | 75 --> 57% |
| Alpha = 0.05 | | |
| Power = 0.8 | 186 | 110 |
| Power = 0.9 | 244 | 144 |
| Alpha = 0.01 | | |
| Power = 0.8 | 271 | 159 |
| Power = 0.9 | 341 | 199 |
| Expected reduction = 50% | 60 --> 30% | 75 --> 37.5% |
| Alpha = 0.05 | | |
| Power = 0.8 | 49 | 32 |
| Power = 0.9 | 63 | 40 |
| Alpha = 0.01 | | |
| Power = 0.8 | 70 | 45 |
| Power = 0.9 | 86 | 55 |
| Expected reduction = 75% | 60 --> 15% | 75 --> 19% |
| Alpha = 0.05 | | |
| Power = 0.8 | 22 | 15 |
| Power = 0.9 | 27 | 18 |
| Alpha = 0.01 | | |
| Power = 0.8 | 30 | 21 |
| Power = 0.9 | 37 | 25 |

Note: all the calculations are for two-sided tests.

COX-2 Inhibitors. COX-2 inhibitors are known in the art and disclosed, for example, in U.S. Pat. Nos. 6,245,797, 6,242,493, 6,235,764, 6,231,888, 6,222,048, 6,211,210, 6,211,189, 6,197,826, 6,136,831, 6,133,292, 6,071,954, 6,057,319, 6,046,217, 6,004,950, 5,994,379, 5,968,958, 5,925,631, 5,861,419, 5,817,700, 5,789,413, 5,733,909, 5,710,140, 5,698,584, 5,691,374, 5,639,780, 5,604,253, 5,550,142, 5,536,752, and 5,521,213, the contents of which are incorporated herein by reference. Specific COX-2 inhibitors can have many fewer side effects than other commonly used NSAIDS, which inhibit both COX-1 and COX-2. A selective inhibitor of cyclooxygenase-2 will have similar anti-inflammatory, antipyretic and analgesic properties to a conventional non-steroidal anti-inflammatory drug, and in addition would inhibit hormone-induced uterine contractions and have potential anti-cancer effects, but will have a diminished ability to induce some of the mechanism-based side effects. In particular, such a compound should have a reduced potential for gastrointestinal toxicity, a reduced potential for renal side effects, a reduced effect on bleeding times and possibly a lessened ability to induce asthma attacks in aspirin-sensitive asthmatic subjects. U.S. Pat. No. 6,222,048. Two particular COX-2 inhibitors are COX-2 inhibitors, Celebrex® (Celacoxib, Searle) and Vioxx® (Rofecoxib, Merck).

COX-2 Inhibitor Administration. Administration of the COX-2 inhibitors are well known in art and disclosed, for example, in U.S. Pat. Nos. 6,245,797, 6,242,493, 6,235,764, 6,231,888, 6,222,048, 6,211,210, 6,211,189, 6,197,826, 6,136,831, 6,133,292, 6,071,954, 6,057,319, 6,046,217, 6,004,950, 5,994,379, 5,968,958, 5,925,631, 5,861,419, 5,817,700, 5,789,413, 5,733,909, 5,710,140, 5,698,584, 5,691,374, 5,639,780, 5,604,253, 5,550,142, 5,536,752, and 5,521,213, the contents of which are incorporated herein by reference. For the treatment, the COX-2 inhibitors may be administered orally, topically, parenterally, by inhalation spray or rectally in dosage unit formulations containing conventional non-toxic pharmaceutically acceptable carriers, adjuvants and vehicles. The pharmaceutical compositions containing the COX-2 inhibitor may be in a form suitable for oral use, for example, as tablets, troches, lozenges, aqueous or oily suspensions, dispersible powders or granules, emulsions, hard or soft capsules, or syrups or elixirs. Corapositions of COX-2 inhibitor intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients that are suitable for the manufacture of tablets. Guidance for the administration can be found in the U.S. Food and Drug approvals for the administration of Celacoxib or Rofecoxib. The COX-2 inhibitors are preferably administered at a dosage amount of about 0.01 to 200 mg/kg of body weight of the patient, preferably about 0.1 to 100 mg/kg of body weight per day.

Also, a method providing for the colonic delivery or preferential metabolism of a COX-2 inhibitor is provided in U.S. Pat. No. 6,231,888.

COX-2 Inhibitor Effectiveness. The effectiveness of the method of the invention can be readily determined by an interview and examination of the patient. A reduction in nocturia and dysuria urgency (for example, an improvement in the patient from level GR II to GRI, or from GR I to normal) is used as a determination that the treatment is effective for the patient. More generally, an analysis of the indices of patient improvement in groups of patients using standardized test (see, FIG. 1 and FIG. 2) can be used to determine that the method of the invention is effective generally.

Also the effectiveness of the method of the invention can be assayed using the reduction of acute phase response indicator markers in the patients blood as compared with untreated patients and control persons. Cengiz M et al., Int. J. Radiat. Oncol. Biol. Phys. 49(4): 1093-6 (2001) has shown that acute phase response is characterized by changes in the plasma concentrations of a number of liver-synthesized proteins, one of which is C-reactive protein (CRP). The existence of these changes in the plasma profile underlies the change in erythrocyte sedimentation rate (ESR). CRP level and ESR increase during radiotherapy and whether their rise correlates with acute and late radiation morbidity. The increase was more prominent in patients who were irradiated through pelvic-paraaortic field than in patients with pelvic radiation. Likewise, the indicator markers can be an increase of RM3/1-positive macrophages, as shown by Handschel J et al., J. Pathol. 193(2): 242-7 (2001) for radiation-induced oral mucositis.

The effectiveness of the method of the invention can be assayed by testing the prostate volume of (male) patients as compared with untreated patients and control persons, using the methods of analysis described by Speight J L et al., Int. J. Radiat. Oncol. Biol. Phys. 48(5): 1461-7 (2000). Likewise, rectal toxicity can be measured using the methods of Hovdenak N et al., Int. J. Radiat. Oncol. Biol. Phys. 48(4): 1111-7 (2000). Intestinal inflammatory response can be tested using the methods of Freeman S L et al., Int. J. Radiat. Biol. 77(3) 389-95 (2001).

Moreover, the method of the invention can be tested in animal models, such as the rat model for radiation-induced proctitis of Kang S et al, J. Korean Med. Sci. 15(6): 682-9 (2000).

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

We claim:

1. A method of reducing one or more deleterious side effect of radiation treatment in a subject undergoing radiation treatment, comprising administering to said subject a side-effect reducing amount of a selective cyclooxygenase-2 inhibitor.

2. The method of claim 1, wherein the cyclooxygenase-2 inhibitor is rofecoxib.

3. The method of claim 1, wherein the cyclooxygenase-2 inhibitor is celecoxib.

4. The method of claim 1, wherein the reduced side effect comprises an acute mucosal effect of radiation on the urinary or gastrointestinal tract.

5. The method of claim 1, wherein the side effect reduced comprises fatigue.

6. The method of claim 1, wherein the reduced side effect comprises a disorder selected from the group consisting of diarrhea, rectal bleeding, proctitis, and sigmoiditis.

7. The method of claim 1, wherein the reduced side effect comprises urinary frequency, prostatitis, or cystitis.

8. The method of claim 1, wherein the radiation treatment is directed outside the pelvis.

9. The method of claim 1, wherein the reduced side effect comprises dermatitis.
